(19) United States
(12) Patent Application Publication
(10) Pub. No.: US 2017/0027758 A1
(43) Pub. Date: Feb. 2, 2017

(54) EAR PLUG DEVICE SYSTEM

(71) Applicant: Glenn R. Hood, III, Columbia, SC (US)

(72) Inventor: Glenn R. Hood, III, Columbia, SC (US)

(21) Appl. No.: 15/222,475

(22) Filed: Jul. 28, 2016

Related U.S. Application Data
(60) Provisional application No. 62/198,098, filed on Jul. 28, 2015.

(51) Int. Cl. A61F 11/08

(52) U.S. Cl. CPC A61F 11/08; A61F 2011/085

(57) ABSTRACT

An earplug device system having three main parts that include the flat attenuation filter, the non-linear filter, and the selective orifices. All three parts work together to create a unique sound experience for users.

### CROSS-REFERENCE TO RELATED APPLICATIONS

The present Utility patent application claims priority benefit of the U.S. provisional application for patent Ser. No. 62/198,098 entitled “MUSICIAN EARPLUG TRAINER” filed 28 Jul. 2015 under 35 U.S.C. 119(e). The contents of this related provisional application are incorporated herein by reference for all purposes to the extent that such subject matter is not inconsistent herewith or limiting hereof

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to an earplug device system. More particularly, certain embodiments of the invention relates to a sound filtering device system.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It may be contemplated that a musician's hearing may be as much of an instrument as an instrument that may play be played by the musician. It may be further contemplated that musicians may routinely be in loud sound environments, wherein less that 1% of musicians may use some form of hearing protection, such as earplugs. It may also be contemplated that many musicians may suffer hearing damage and/or Noise Induced Hearing Loss and thus one aspect of conventional hearing protection may be that the only hearing protection that may work may be a hearing protection that a person may actually use. Conventional hearing protection may block substantially all perceivable, unperceivable, damaging, and non-damaging sound waves from entering a user's ear canal, and thus may effectively prevent the user from hearing sound that may be very loud and also may prevent the user from hearing sound that may be very soft and/or quiet. For example, in a case that a user may be in a transitioning sound level environment, such as a concert hall, the user may not be able to hear very loud sounds and may also not be able to hear quieter sounds such as subtle cue changes in their own instrument or instruments around them. The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that conventional earplugs that offer a form of hearing protection may utilize a select filter. It may be contemplated that when in use, there may be an occluded feeling felt by the wearer and the earplug may completely reduce outside sound by 9, 15, or 25 dB, (dB is an algorithmic formula). It may be further contemplated that this reduction may make it difficult for the wearer to be able to hear and understand desirable low volume sound also included in the outside sound because of occlusion and distortion This effect may be directly due to a foreign impact on pitch, volume, and clarity which may be drastically different as compared to when no hearing protection is utilized. It may be contemplated that because of this effect, a user may opt to forego usage of hearing protection. In a transitional sound level environment, one conventionally considered approach to account for these environments may be for the wearer to stop current activity to take out an earplug being worn as the sound level of the environment changes from loud to soft. It may be contemplated that such approaches may not provide convenience, and may further cause a user may to forego usage of hearing protection.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1A through FIG. 1C illustrate various exemplary views of exemplary components of an embodiment of a sound filtering device system, in accordance with an embodiment of the present invention, where FIG. 1A illustrates an outline view thereof, FIG. 1B illustrates a transparent view thereof, and FIG. 1C illustrates a textured view thereof;

FIG. 2 illustrates various views of an exemplary container portion of an embodiment of a sound filtering device system, in accordance with an embodiment of the present invention;

FIG. 3, illustrates an exemplary configuration of an exemplary sound filtering device embodiment, in accordance with an embodiment of the present invention;

FIG. 4, illustrates another exemplary configuration of an exemplary sound filtering device embodiment, in accordance with an embodiment of the present invention;

FIG. 5, illustrates another exemplary configuration of an exemplary sound filtering device embodiment, in accordance with an embodiment of the present invention;

FIG. 6, illustrates another exemplary configuration of an exemplary sound filtering device embodiment, in accordance with an embodiment of the present invention;

FIG. 7 illustrates an exemplary configuration of an exemplary pair of sound filtering devices, in accordance with an embodiment of the present invention;

FIG. 8 illustrates a flowchart of an exemplary method of using a sound filtering device system embodiment, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognize in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)″ Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit—“about 10%”—the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. §112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention.

Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache. . . . ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. .sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

It may be contemplated that what may be needed may be a device that may allow a user to possibly hear in substantially all types of sound environments, and may still provide protection for the user's hearing. It may be further contemplated that at this time no one else may be sufficiently solving this industry problem. Many others may be attempting to, but the adoption process may be slow. Many embodiments of the present invention may include a device, which may further include a dual filter system, that may not isolate a user completely from all environmental sounds, but may instead take the user's hearing perception into account, by allowing the user to hear substantially all non-harmful sound waves and as environmental sound level may increase, the device system may still block out harmful sound waves. Many embodiments may also provide a device system that may be placed in a user's ear and may change the amount of back pressure a user may experience in their head by possibly providing an open passage along with protection, and thus may allow the user to be fully protected, at times of use, in various environmental sound level situations without the user having to take the device system in and out of the user's ear.

It may be further contemplated that embodiments of the device system may allow a user's hearing to not be completely occluded, while also allowing a user to possibly be able to hear a faintest sound available. This may allow a user to be aware of substantially all minor sound changes while also possibly having their hearing fully protected. In many embodiments the device system may include at least one first filter, at least one second filter and at least one selectable orifice. In some embodiments a user may train their sound perception to a new way of hearing, wherein they may continuously reduce an amount of sound coming through the filters until they may achieve an optimal balance of environmental sound and filtered sound.

It may further be contemplated that many embodiments of the present invention may aid in acclimating a user to a use of hearing protection, wherein some embodiments may acclimate a user to a sound reduction sensations that may occur during a use of sound protection. Many embodiments of the present invention may include a dual filtered hearing protection system which may utilize one or more flat attenuation filters and one or more passive non-linear acoustic filters, while possibly employing a series of small interchangeable sound pressure vent reduction orifices. In some embodiments the one or more non-linear filters may be combined with the interchangeable orifices to possibly provide a training functionality for users. It may be contemplated that in many embodiments, a user may be able to learn to align a mentally perceived sound with an actual acceptable sound produced by a played instrument by using a multiplicity of orifices in conjunction using the flat attenuation filer, wherein the learning may include the user downsizing the orifices while using the flat attenuation filter. In some embodiments, after the learning is complete, the user may be able to use present invention in different configurations and in different environments.

At least one difference, for example, and without limitation, between many embodiments of the present invention and conventional devices may be that embodiments of the present invention may provide a sound filtering device system that may use selective flat attenuation filtering, along with selective passive non-linear filtering with interchangeable orifices for a user to possibly achieve an exact sound attenuation mix that may be desirable to the user. By using the non-linear filter with the flat attenuation filter, this may reduce the occluded feeling while fully protecting the person's hearing. In some embodiments of the present invention, the dual filtered hearing protection system may further include a pop-cord lanyard which may allow a user an ability to remove an inserted system from the user's ears and allow it to hang it around the user's neck. It should be noted that over one thousand sets of the described device system have been sold the marketplace.

As will be described in some detail below with reference to the figures, in many embodiments of the present invention an interchangeable, customizable, sound filtering, hearing protection device system may be utilized by a multiplicity of users, in a variety of different sound environments, such as, and without limitation, musicians, club goers, industrial workers, performance audience participants, subway and other public transportation riders, sound sensitive people, and substantially anyone that may need to reduce loud environment sounds while still possibly being able to hear normal range and soft/quite range sounds.

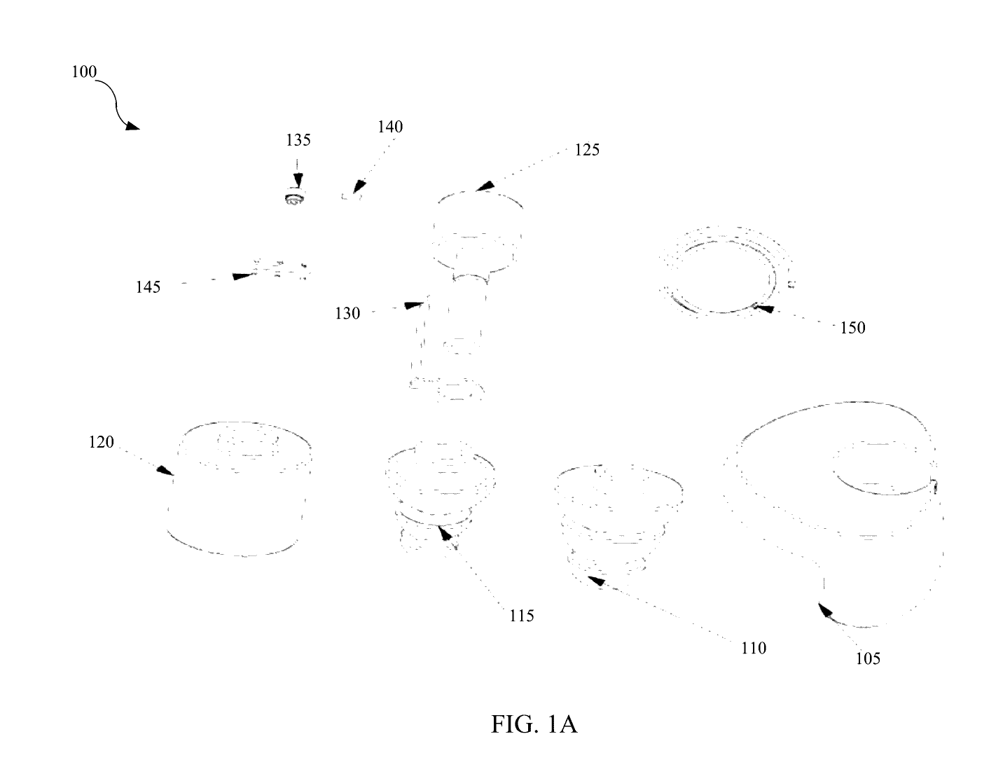

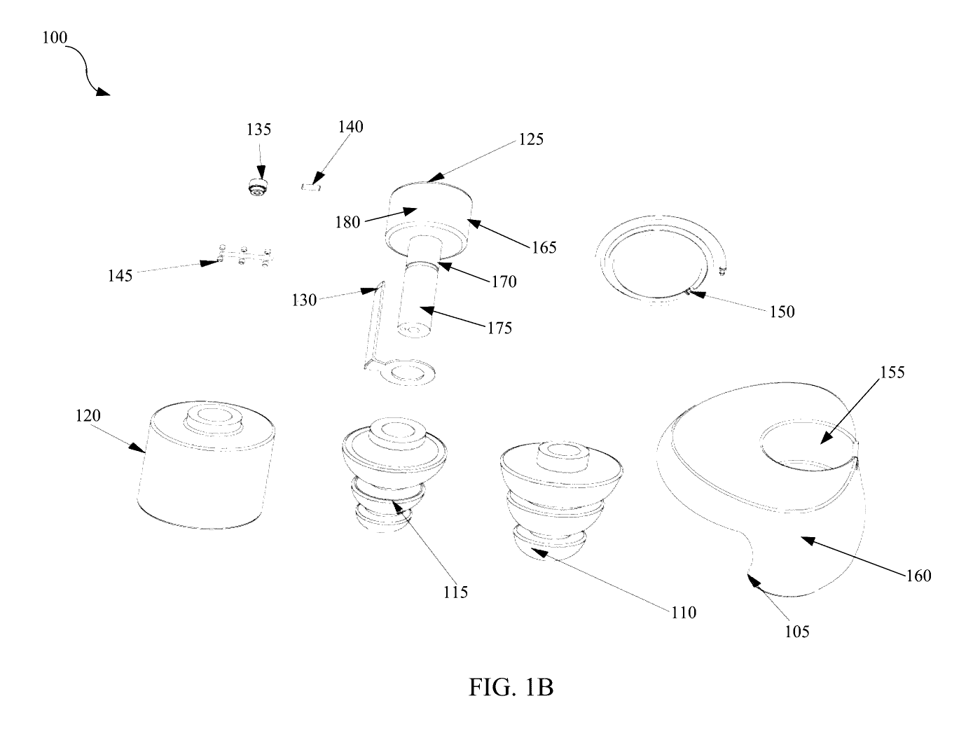

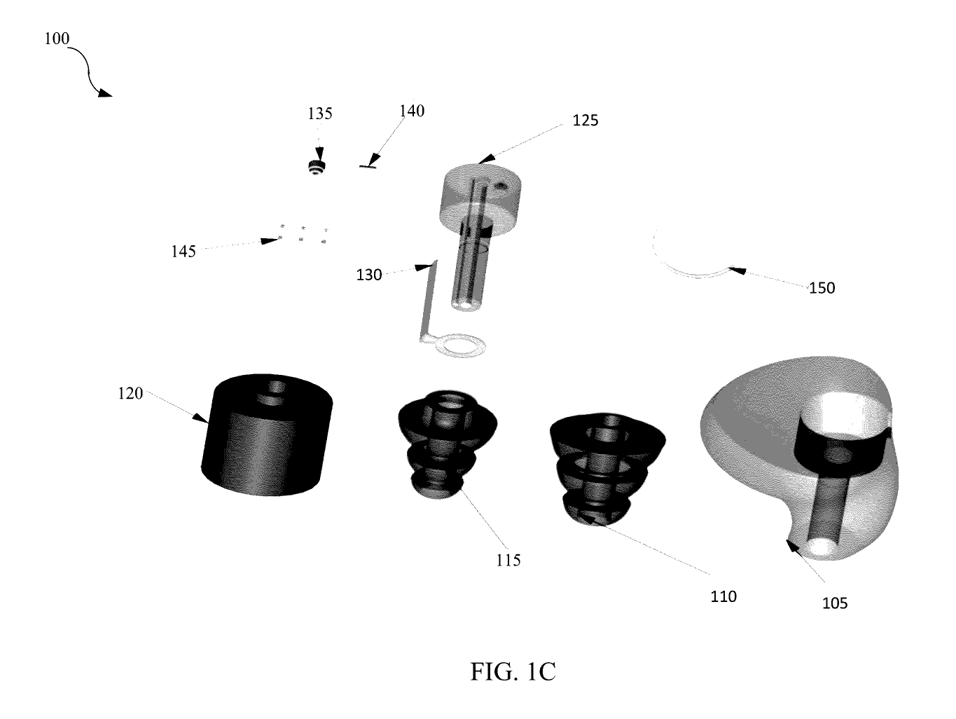

FIG. 1A through FIG. 1C illustrate various exemplary views of exemplary components of an embodiment of a sound filtering device system 100, in accordance with an embodiment of the present invention, where FIG. 1A illustrates an outline view thereof, FIG. 1B illustrates a transparent view thereof, and FIG. 1C illustrates a textured view thereof. In the present embodiment, the sound filtering device system may include a first ear insert portion 105, a second ear insert portion 110, a third ear insert portion 115, a fourth ear insert portion 120, a container portion 125, a pull tab 130, a first filter 135, a second filter 140, a multiplicity of interchangeable orifices 145, and a pop-cord lanyard 150, wherein one or more of the components may be used to configure particular embodiments of a the sound filtering device, in accordance with embodiments of the present invention. Those skilled in the art, in light of and in accordance with the teachings of the present invention, would readily recognize that described features of the embodiments described above and below may be applied for example, and without limitation, to a sound filtering device for use in a user's right ear and a sound filtering device for use in the user's left ear.

In many embodiments of the present invention, first ear insert portion 105 may be a plug, wherein the plug may be configured to be ergonomically shaped to fit into at least one ear canal of a user. In the present embodiment, and with reference to FIG. 1B, the plug may further include a hollow tubular space, wherein a first portion of the hollow tubular space 155 may include a first diameter and a second portion of the hollow tubular space 160 may include a second diameter. Furthermore, the first diameter may be larger than the second diameter. In many embodiments, a material of the first ear insert portion may include, without limitation, silicone, rubber, plastic, acrylic, foam, polyvinyl chloride (PVC), polyurethane (PU), vinyl materials, rolled materials, no-roll materials, any combination thereof, or any material suitable for the needs of a particular application. In some embodiments the first ear insert portion may be custom molded to a user's ear canal. In many embodiments, the hollow tubular space may be configured to house pull tab 130 and container portion 125, wherein the container portion may be removably joined into the hollow tubular space of the first ear insert portion.

In many embodiments of the present invention, container portion 125 may be cylindrical in shape and may further include a material such as, and without limitation, silicone, rubber, plastic, acrylic, foam, polyvinyl chloride (PVC), polyurethane (PU), vinyl materials, metal, ceramic, any combination thereof, or any material suitable for the needs of a particular application. In many embodiments, the container portion may further include a first cylindrical portion and a second cylindrical portion, wherein the first cylindrical portion 165 may include a third diameter and the second cylindrical portion 170 may include a fourth diameter. Furthermore, the third diameter may be approximately equal to the first diameter, and the fourth diameter may be approximately equal to the second diameter. In many embodiments, the container portion may include a first hollow channel 175 and a second hollow channel 180, wherein each hollow channel may extend from a top portion of the container portion to a bottom portion of the container portion. The first hollow channel may include a first top opening in the top portion of the container portion and the first hollow channel may also include a first bottom opening in a bottom portion of the container portion. Moreover, the second hollow channel may include a second top opening in the top portion of the container portion and the second hollow channel may also include a second bottom opening in the bottom portion of the container portion. In some embodiments of the present invention the top portion of container portion 125 may further include a third top opening 185, wherein the third top opening may include a pop-cord lanyard hole.

In many embodiments of the present invention, first filter 135 may be cylindrical in shape and may further include a fifth diameter, wherein the first filter may also include a first internal hollow passage. In some embodiments, the first filter may be removably joined into first hollow channel 175, wherein the first filter may be a flat attenuation filter that may attenuate environmental sound waves above a particular decibel. The flat attenuation filter comes in interchangeable dB reduction frequencies from 4 decibel reduction to 27 decibel reduction based on the filter selected.

The reduction is achieved by using a proprietary layered frequency mesh that will reduce the sound as it goes through the flat attenuation filter. Somewhat like a layered screen door. The more layers, the more reduction.

In many embodiments of the present invention, second filter, 140 may be cylindrical in shape and may further include a sixth diameter, wherein the second filter may also include a second internal hollow passage. In some embodiments, the second filter may be removably joined into second hollow channel 180, wherein the second filter may be a passive non-linear filter that may attenuate environmental sound waves non-linearly. In some embodiments, the non-linear filter allows the smallest sound to travel through the open non-linear filter until 85 dB is achieved. Once sound pressure reaches 85 dB all higher sounds are attenuated by the filter. For example, hooking of a 4″ fire hose to a 1″ garden hose. As the fire hydrant is gently opened, water flows freely through the garden hose. As it opens further, and pressure is increased, the water is limited to the garden hose no matter how much the fire hydrant is opened.

Another embodiment may include any combination of the following, without limitation, silicone, rubber, plastic, acrylic, foam, polyvinyl chloride (PVC), polyurethane (PU), vinyl materials, rolled materials, no-roll materials, any combination thereof, or any material suitable for the needs of a particular application.

Furthermore, in many embodiment of the present invention, a particular orifice from a multiplicity of interchangeable orifices 145, may be selected and used in conjunction with the second filter, wherein orifices of the multiplicity of orifices may be sound pressure vent reduction orifices. By placing the smaller orifice vent individually in each side the user may achieve a reduction in the amount of sound that may come through the non-linear filter. There are four different selections of reduction from full open (no orifice) to 30% restricted orifice, 70% restricted orifice to a full plug of the nonlinear filter. This is achieved by placing the selected orifice in the vent opening of the nonlinear filter.

In many embodiments, the first filter, the second filter, and at least one particular orifice of the multiplicity of orifices may work together to create the unique sound experience. Furthermore, the selection of a series of interchangeable orifices may help train a user's ear to a new way of hearing by each orifice possibly being able to progressively reduce the amount of sound coming through the second filter as each orifice is inserted until the user may achieve an optimal balance of perceived environmental sound and filtered sound, wherein the optimal balance may further include the user only needing to use the first filter in loud situations.

In some embodiments, an exemplary, and without limitation user, in a music class, depending on the day and the position the user is sitting in, the user can select the flat attenuation filter to hear, for instance, more in user's left ear, less in the right ear; or reduce more in the user's right ear to achieve a balance so when the user is playing an instrument the user hears the true color of the music in their head while playing.

Furthermore, in some embodiments, an exemplary, and without limitation user, may be a musician playing different instruments. In this exemplary, and without limitation, case, by using different combinations of components, the user may be able to play the different instruments as needed while reducing and varying a back pressure by changing particular components of sound filtering devices that may be inserted into one or both of the user's ear canals.

In many embodiments of the present invention, pop-cord lanyard 150 may be removably joined to container portion 125 by being joined to the third top opening using a joining mechanism, such as, and without limitation, a snap fit configuration of the third opening and a pop-cord lanyard end, an adhesive, a threading configuration of the third opening and a pop-cord lanyard end, or any suitable joining mechanism for the needs of a particular application. In some embodiments, the pop-cord lanyard may comprise a material such as, and without limitation, silicone, rubber, plastic, acrylic, foam, polyvinyl chloride (PVC), polyurethane (PU), vinyl materials, metal, ceramic, polyester, nylon, satin, silk, cotton, polyethylene terephthalate (PET), braided leather, braided para-cord, any combination thereof, or any material suitable for the needs of a particular application, or any combination thereof. In some embodiments, it may be desirable to join the pop-cord lanyard into the third opening in quiet situations and in a case that the environment may be excessively loud, the pop-cord lanyard may be joined to the second top opening to possibly achieve a maximum hearing attenuation. In another example, there may be some situations, for example, but not limited to a percussion rehearsal, whereas the musician may take the pop-cord from the pop-cord top hole and place it in the non-linear filter hole. This will then only allow sound to come through the flat attenuation filter. The reason for this is that in a drum rehearsal everything is loud and there is no need to hear very quiet sounds.

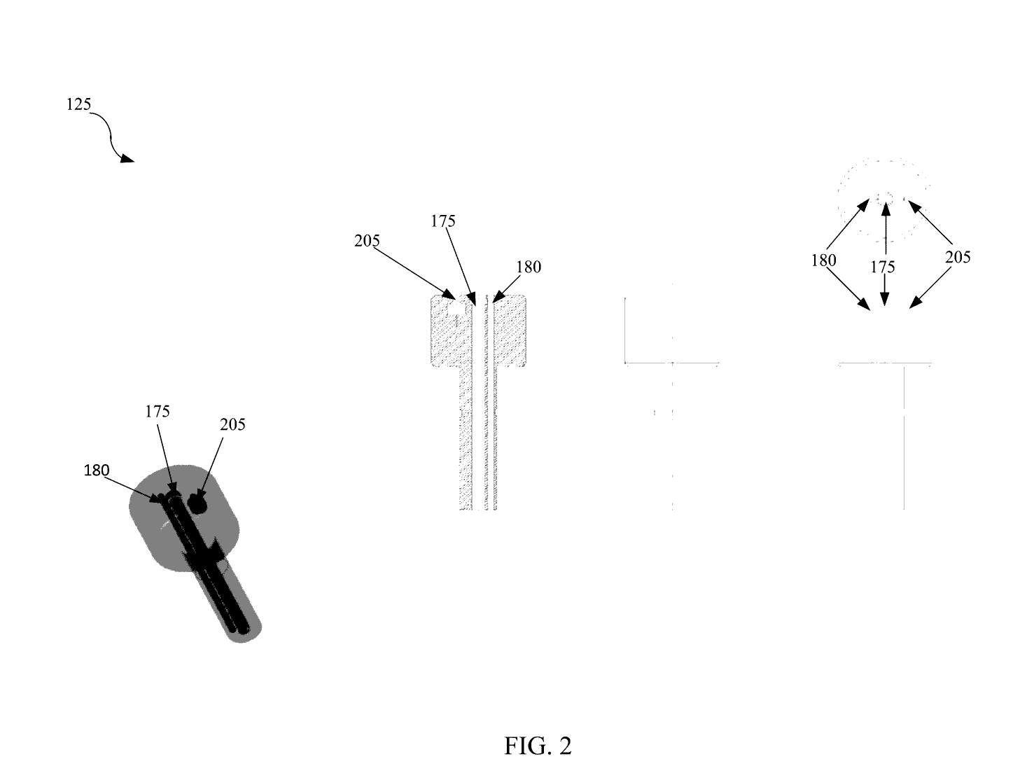

FIG. 2 illustrates various views of an exemplary container portion 125 of an embodiment of a sound filtering device system, in accordance with an embodiment of the present invention. In many embodiments of the present invention, and with reference to FIG. 1B, the first hollow channel 175 may be of seventh diameter, and the second hollow channel 180 may be of an eighth diameter, wherein the seventh diameter may be larger than the eighth diameter. In some embodiments, the first hollow channel may be centrally located within container portion 125, while the second hollow channel may be off-centrally located within container portion 125. In some alternative embodiments, the first hollow channel may be off-centrally located within a container portion, while the first hollow channel may be centrally located within a container portion. Furthermore, in some embodiments, container portion 125 may be substantially solid except for spaces of the first hollow channel, the second hollow channel, and the third top opening 205. In some alternative embodiments a container portion may include only a first hollow channel. In some other alternative embodiments a container portion may include only a second hollow channel. In yet other alternative embodiments a container portion may include three or more hollow channels, wherein three or more filters may be utilized by joining one filter to each hollow channel. In another embodiment a multiplicity of filters may be joined into a first hollow channel and/or a multiplicity of filters may be joined into a second hollow channel.

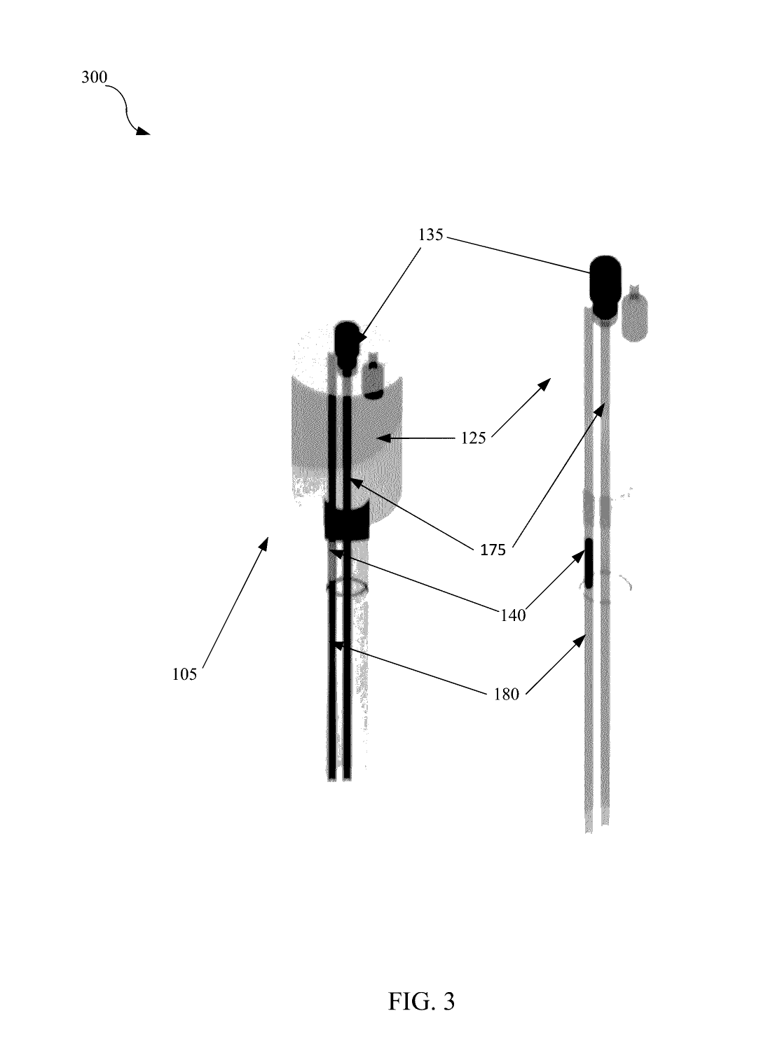

FIG. 3, illustrates an exemplary configuration of an exemplary sound filtering device embodiment 300, in accordance with an embodiment of the present invention. In many embodiments, and with reference to FIG. 1B, the container portion 125 may be removably joined to the first ear insert portion 105 by being inserted into the hollow tubular spaces of the container portion. Furthermore first filter 135 may be removably joined to first hollow channel 175 and second filter 140 may be removably joined to second hollow channel 180.

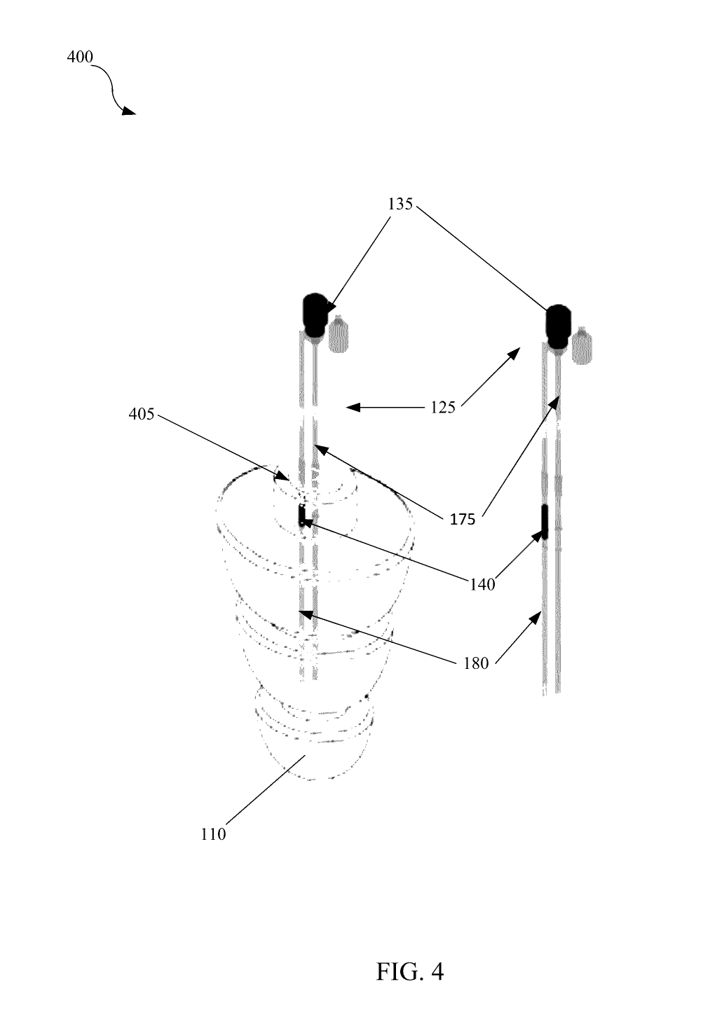

FIG. 4, illustrates another exemplary configuration of an exemplary sound filtering device embodiment 400, in accordance with an embodiment of the present invention. In many embodiments of the present invention, and with reference to FIG. 1B, second ear insert portion 110 may be a flange plug, wherein the plug may be configured to include a multiplicity of flanges and may further fit into at least one ear canal of a user. In the present embodiment, the flange plug may further include a flange plug hollow tubular space 405, wherein the flange plug hollow tubular space may include a ninth diameter, wherein the ninth diameter may be approximately equal to the fourth diameter. In many embodiments, a material of the second insert ear portion may include, without limitation, silicone, rubber, plastic, acrylic, foam, polyvinyl chloride (PVC), polyurethane (PU), vinyl materials, rolled, no-roll, any combination thereof,], or any material suitable for the needs of a particular application. In many embodiments, the flange plug hollow tubular space may be configured to house container portion 125, wherein the container portion may be removably joined into the flange plug hollow tubular space of the second insert ear portion by inserting the second cylindrical portion of the container portion into the flange plug hollow tubular space. Furthermore first filter 135 may be removably joined to first hollow channel 175 and second filter 140 may be removably joined to second hollow channel 180.

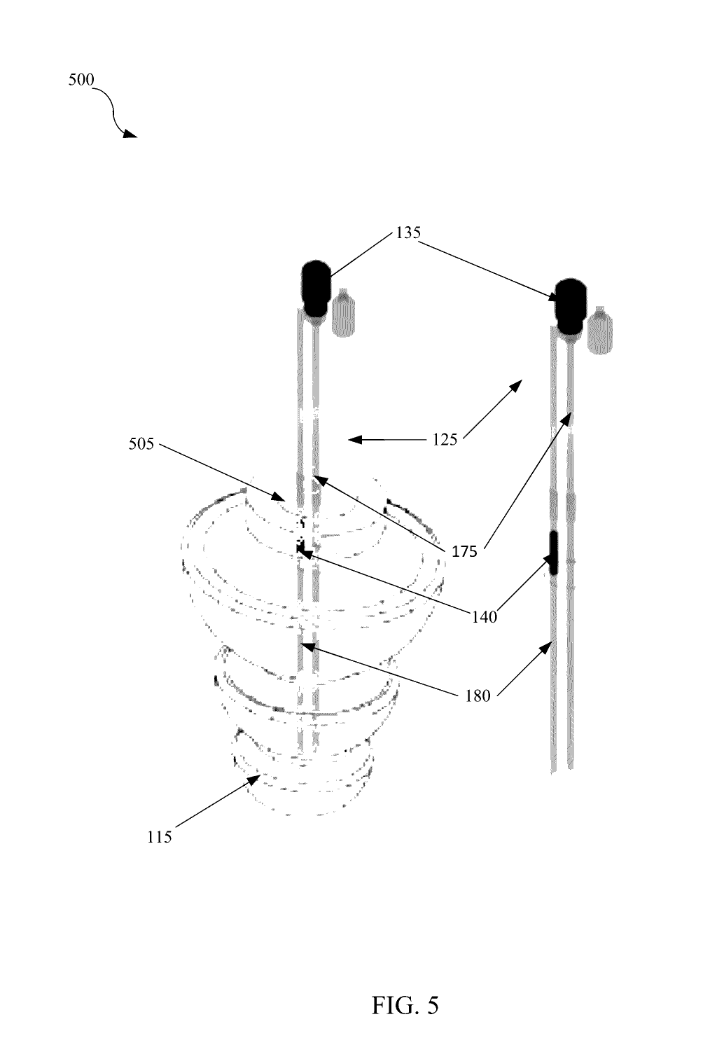

FIG. 5, illustrates another exemplary configuration of an exemplary sound filtering device embodiment 500, in accordance with an embodiment of the present invention. In many embodiments of the present invention, third ear insert portion 115 may be a small flange plug, wherein the small flange plug may be configured to include a multiplicity of flanges and may further fit into at least one ear canal of a user. In the present embodiment, and with reference to FIG. 1B, the small flange plug may further include a small flange plug hollow tubular space 505, wherein the flange plug hollow tubular space may include a tenth diameter, wherein the tenth diameter may be approximately equal to the fourth diameter. In many embodiments, a material of the third insert ear portion may include, without limitation, silicone, rubber, plastic, acrylic, foam, polyvinyl chloride (PVC), polyurethane (PU), vinyl materials, rolled, no-roll, any combination thereof, or any material suitable for the needs of a particular application. In many embodiments, the small flange plug hollow tubular space may be configured to house container portion 125, wherein the container portion may be removably joined into the small flange plug hollow tubular space of the third insert ear portion by inserting the second cylindrical portion of the container portion into the small flange plug hollow tubular space. Furthermore first filter 135 may be removably joined to first hollow channel 175 and second filter 140 may be removably joined to second hollow channel 180.

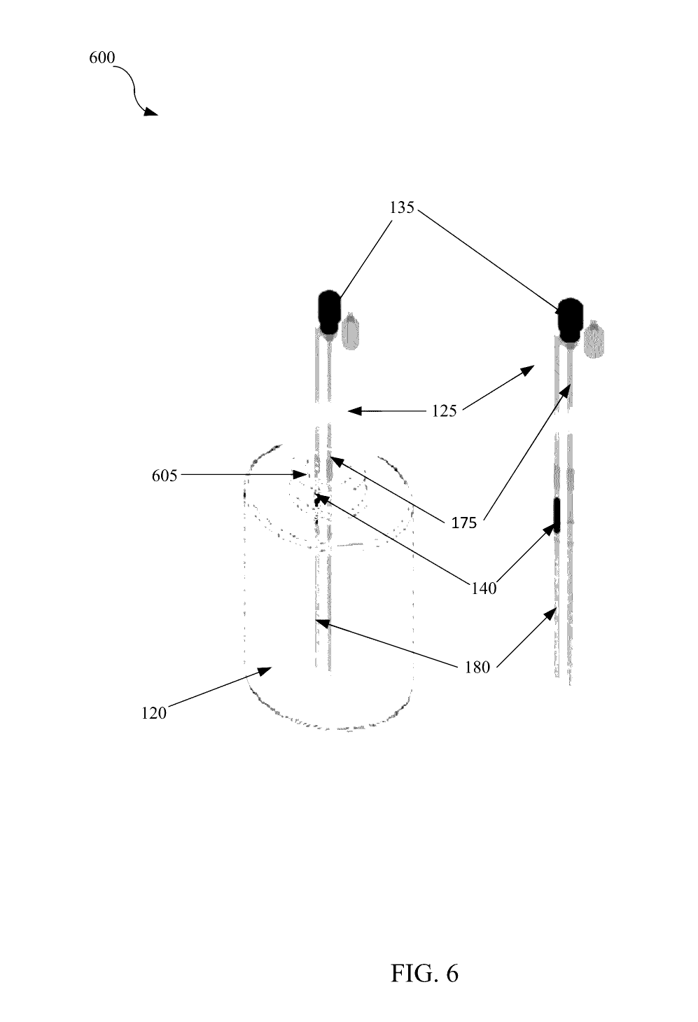

FIG. 6, illustrates another exemplary configuration of an exemplary sound filtering device embodiment 600, in accordance with an embodiment of the present invention. In many embodiments of the present invention, fourth ear insert portion 120 may be a foam compliance tip plug, wherein the foam compliance tip plug may be configured to be cylindrical in shape and may further fit into at least one ear canal of a user. In the present embodiment, and with reference to FIG. 1B, the foam compliance tip plug may further include a foam compliance tip plug hollow tubular space 605, wherein the foam compliance tip plug hollow tubular space may include an eleventh diameter, wherein the eleventh diameter may be approximately equal to the fourth diameter. In many embodiments, a material of the fourth insert ear portion may include, without limitation, foam, polyvinyl chloride (PVC), polyurethane (PU), rolled foam composites, no-rolled foam composites, any combination thereof, or any other foam material suitable for the needs of a particular application. In many embodiments, the foam compliance tip plug hollow tubular space may be configured to house container portion 125, wherein the container portion may be removably joined into the foam compliance tip plug hollow tubular space of the fourth insert ear portion by inserting the second cylindrical portion of the container portion into the foam compliance tip plug hollow tubular space. Furthermore first filter 135 may be removably joined to first hollow channel 175 and second filter 140 may be removably joined to second hollow channel 180.

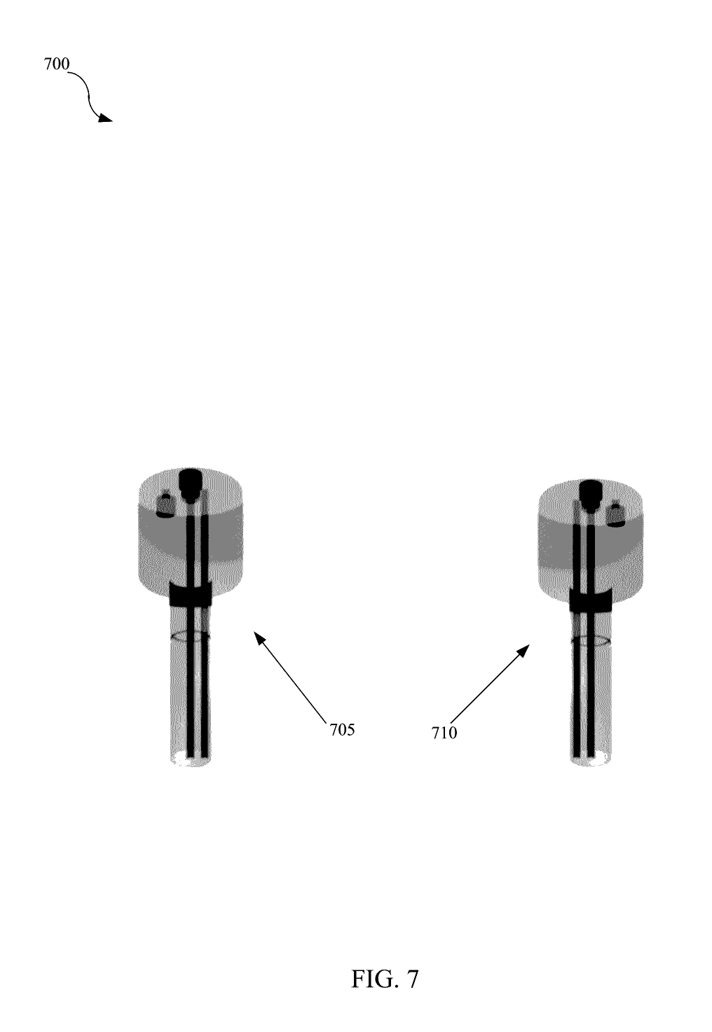

FIG. 7 illustrates an exemplary configuration of an exemplary pair of sound filtering devices 700, in accordance with an embodiment of the present invention. In many embodiments of the present invention, a pair of sound filtering devices may be used by a user, wherein a first sound filtering device 705 may be inserted into a first ear canal of a user and a second sound filtering device 710 may be inserted into a second ear canal of the user. With reference to FIG. 4 through FIG. 6, each sound filtering device 400, 500, and 600, may be used, by a user, with a respective mirror image device, as a pair of sound filtering devices, wherein one of the sound filtering device may be inserted into a first ear canal and another sound filtering device may be inserted into a second ear canal. In many embodiments, by interchangeably combining use of the first filter, the second filter, and the particular orifice of the multiplicity of orifices, a user may be able to alter hearing attenuation qualities of each ear independently. For example, and without limitation, in a case that the environment may be louder on a right side of the user, the user may reduce sound more, and/or in a case that the environment may be quieter on a left side of the user, the user may reduce sound less. In an alternative embodiment a pair of sound filtering devices may include non-matching ear insert portions as well as different combinations of filters and orifices being used for each ear.

In other embodiments of the present invention, the first filter and second filter may also be interchangeable with each other and other filters of the same type. It may be contemplated that being able to interchange the first filters and the second filters in a one size fits all sound filtering device may be very beneficial. Furthermore, in yet some other embodiments, a shape, color, and design of a sound filtering device may be substantially any shape, color, and design, suitable for the needs of a particular application.

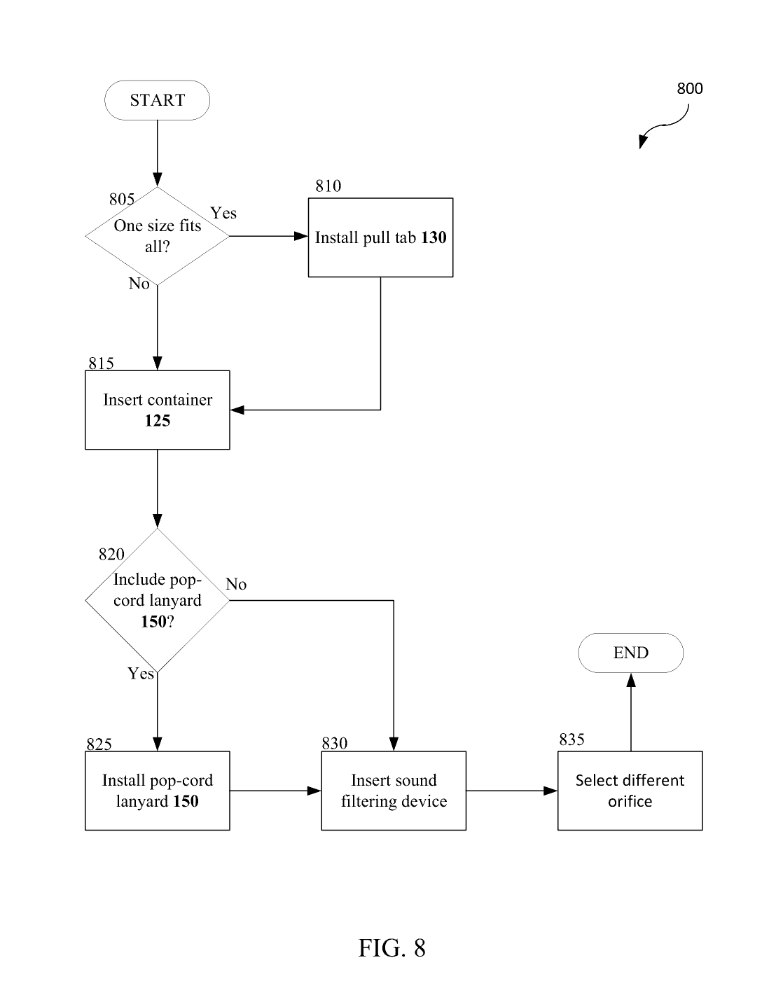

FIG. 8 illustrates a flowchart of an exemplary method 800 of using a sound filtering device system embodiment, in accordance with an embodiment of the present invention. In the present embodiment, and with reference to FIG. 1A, and FIG. 2, the method of using a sound filtering device system may begin with a user possibly choosing an ear insert portion to be placed into the user's ear canal in a decision step 805, by selecting a “one size fits all solution” or not, wherein the one size fits all solution may be the first ear insert portion 105 and other solutions may be one of second ear insert portion 110, third ear insert portion 115, and fourth ear insert portion 120. The user may then install pull tab 130 into first ear insert portion 105 in a step 810 in a case that the user chose the “one size fits all solution”. In a case that the user may not choose the “one size fits all solution” and/or the pull tab may already be installed in the first ear insert portion, the user may insert container 125, with inserted first filter, second filter, and selected orifice, into the chosen ear insert portion in a step 815. Next, a user may choose to include pop-cord lanyard 150, in a step 820. In a case that the user may choose to include the pop-cord lanyard, the pop-cord lanyard may be installed into the third top opening 215, in a step 825. In a case that the user may choose not to use pop-cord lanyard and/or the pop-cord lanyard may already be installed, in a step 830, a user may insert the sound filtering device (as illustrated in FIG. 3 through FIG. 6) into the user's ear canal for use. After use for some time, the user may become acclimated to the sound filtering device, and in a step 835, the user may then select another orifice to use to possibly modify the amount of sound that may be allowed though the non-linear filter. The user may continue selecting orifices until an optimal balance of environmental sound and filtered sound may be achieved.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing an earplug sound filtering device system according to the present invention will be apparent to those skilled in the art. Various aspects of the invention have been described above by way of illustration, and the specific embodiments disclosed are not intended to limit the invention to the particular forms disclosed. The particular implementation of the earplug sound filtering device system may vary depending upon the particular context or application. By way of example, and not limitation, the earplug sound filtering device system described in the foregoing were principally directed to customizable, and interchangeable earplugs, filters, and orifices that may be used in combination or alone to help attenuate loud environmental sound while also possibly training and enabling a user to hear faint/soft sound. implementations; however, similar techniques may instead be applied to motorcycle helmets and/or hard hats, to help hear radio communications while protecting a user's hearing, hearing protection for all employees in the music field, concert venues, stadiums, & sports events that are covered under the OSHA and state guidelines, hearing protection for pilots, astronauts and even swimmers and divers to help restrict a water flow into the ears and further help maintain a back pressure which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

What is claimed is:

1. An apparatus for an earplug device system comprising:
Means for the use of a combination of interchangeable flat attenuation filters and interchangeable non-liner filters;
Means for selective orifices to decrease the amount of sound that will come through the open non-linear filter, and gradually adjust the perception of your hearing to be able to over time adjust to only using the flat attenuation filter in loud situations;
Means for using the device in a quiet situation in the pop-cord lanyard hole, and when necessary in extremely loud situations placed in the non-linear filter hole to achieve maximum hearing attenuation;
Means for the use of flat attenuation filters and non-linear filters in combination, along with reducing orifices can give you independent ear attenuation qualities.
Means for different combinations allowing user to play different instruments as the need for reduction and back varies with each instrument user plays' and
Means for the ability to interchange the flat attenuation and non-linear filters in the device with other user products.
